(19) United States
(12) Patent Application Publication
(10) Pub. No.: US 2022/0362326 A1
(43) Pub. Date: Nov. 17, 2022

(54) INHIBITING ZD17-JNK INTERACTION AS A THERAPY FOR ACUTE MYOCARDIAL INFARCTION

(71) Applicant: The University of British Columbia, Vancouver (CA)

(72) Inventors: Jack Wuyang Jin, Vancouver (CA); Yu Tian Wang, Vancouver (CA); Max S. Cynader, Vancouver (CA)

(73) Assignee: The University of British Columbia, Vancouver, BC (CA)

(21) Appl. No.: 17/642,936

(22) PCT Filed: Sep. 11, 2020
(86) PCT No.: PCT/CA2020/051229

§ 371 Date: Mar. 14, 2022

Related U.S. Application Data
(60) Provisional application No. 62/899,440, filed on Sep. 12, 2019.

(51) Int. Cl. A61K 38/08; A61K 47/64; A61P 9/10

(52) U.S. Cl. CPC A61K 38/08; A61K 47/645; A61P 9/10

(57) ABSTRACT

Disclosed herein are uses of a polypeptide comprising NIMoEsh to treat a disease or condition associated with acute myocardial infarction (AMI) in a subject in need thereof, of a polypeptide comprising NIMoEsh to restore heart function after AMI in a subject in need thereof, of a polypeptide comprising NIMoEsh to reduce or prevent AMI-induced heart function loss in a subject in need thereof, of a polypeptide comprising NIMoEsh to reduce AMI-induced heart tissue infarct in a subject in need thereof, and of a polypeptide comprising NIMoEsh to protect cardiomyocytes against AMI-induced function loss in a subject in need thereof. Disclosed also herein are methods by which such treating, restoring, reducing or preventing, reducing, and/or protecting may be done, and a polypeptide comprising NIMoEsh for use in such treating, restoring, reducing or preventing, reducing, and/or protecting.

### CROSS REFERENCE TO RELATED APPLICATION

This application claims priority to U.S. Provisional Patent Application Ser. No. 62/899,440 filed on Sep. 12, 2019.

### FIELD OF THE INVENTION

The present disclosure relates generally to cardioprotection, and more particularly to using polypeptides to treat acute myocardial infarction.

### BACKGROUND TO THE DISCLOSURE

Acute myocardial infarction (AMI), commonly known as heart attack, is a life-threatening cardiovascular disease. During AMI, blood supply to the heart is suddenly limited due to coronary artery blockade, which can result in severe damage to the heart. AMI causes more than 2.4 million deaths in the USA, more than 4 million deaths in Europe and northern Asia, and more than a third of deaths in developed nations annually. (Reed, G. W., Rossi, J. E. & Cannon, C. P. Acute myocardial infarction. Lancet 389, 197-210, doi:10.1016/50140-6736(16)30677-8 (2017))

Use of current medications, such as aspirin, nitroglycerin and statin, have been reported to help reduce the risk of AMI (see, e.g., Ferreira, J. C. & Mochly-Rosen, D. Circ J 76, 15-21 (2012); Dai, Y. & Ge, J. Thrombosis 2012, 245037, doi:10.1155/2012/245037 (2012); Fung, V., et al. PLoS One 13, e0191817, doi:10.1371/journal.pone.0191817 (2018)), but not prevent death of cardiomyocytes after AMI. Similarly, current surgical approaches, such as percutaneous coronary intervention and coronary artery bypass surgery (Perrier, S., et al. Interact Cardiovasc Thorac Surg 17, 1015-1019, doi:10.1093/icvts/ivt381 (2013)), can help restore blood circulation to the heart, but have not been reported to have cardioprotective efficacy after AMI.

To date, the FDA has not approved a cardioprotective drug for AMI. For example, cyclosporine, a small molecule that showed potential cardioprotective efficacy in a variety of animal AMI models, failed in human clinical trials. (Rahman, F. A. et al. Efficacy and Safety of Cyclosporine in Acute Myocardial Infarction: A Systematic Review and Meta-Analysis. Front Pharmacol 9, 238, doi:10.3389/fphar.2018.00238 (2018))

Despite the advances made to date in the development of cardioprotective treatment, there is room for improvement to address the above-mentioned problems and shortcomings of the prior art.

### SUMMARY OF THE INVENTION

It is an object of the present invention to obviate or mitigate at least one of the above-mentioned disadvantages of the prior art.

It is another object of the present invention to provide a novel therapy for cardioprotection.

Accordingly, in one of its aspects, the present disclosure provides a method of treating a disease or condition that is associated with, or is, acute myocardial infarction (AMI) in a subject in need thereof, the method comprising administering a therapeutically effective amount of a polypeptide comprising NIMoEsh to the subject.

In another of its aspects, the present disclosure provides a method of restoring heart function after AMI in a subject in need thereof, the method comprising administering a therapeutically effective amount of a polypeptide comprising NIMoEsh to the subject.

In another of its aspects, the present disclosure provides a method of reducing or preventing AMI-induced heart function loss in a subject in need thereof, the method comprising administering a therapeutically effective amount of a polypeptide comprising NIMoEsh to the subject.

In another of its aspects, the present disclosure provides a method of reducing AMI-induced heart tissue infarct in a subject in need thereof, the method comprising administering a therapeutically effective amount of a polypeptide comprising NIMoEsh to the subject.

In another of its aspects, the present disclosure provides a method of protecting cardiomyocytes against AMI-induced function loss in a subject in need thereof, the method comprising administering a therapeutically effective amount of a polypeptide comprising NIMoEsh to the subject.

In another of its aspects, the present disclosure provides a use of a polypeptide comprising NIMoEsh to treat a disease or condition associated with AMI in a subject in need thereof.

In another of its aspects, the present disclosure provides a use of a polypeptide comprising NIMoEsh to restore heart function after AMI in a subject in need thereof.

In another of its aspects, the present disclosure provides a use of a polypeptide comprising NIMoEsh to reduce or prevent AMI-induced heart function loss in a subject in need thereof.

In another of its aspects, the present disclosure provides a use of a polypeptide comprising NIMoEsh to reduce AMI-induced heart tissue infarct in a subject in need thereof.

In another of its aspects, the present disclosure provides a use of a polypeptide comprising NIMoEsh to protect cardiomyocytes against AMI-induced function loss in a subject in need thereof.

In another of its aspects, the present disclosure provides a polypeptide comprising NIMoEsh for one or more of the above uses.

Thus, the present inventors have developed a novel therapy for cardioprotection in a subject. This cardioprotective therapy can be provided after AMI to reduce or prevent AMI-induced damage to heart tissue. In particular, through use of this therapy, cardiomyocytes may be protected against AMI-induced cell death or loss of function. Furthermore, heart function, at least in part, may be restored after AMI with use of the present therapy. The present therapeutic use may provide an alternative to existing pharmaceutical and non-pharmaceutical treatment options, and, in particular, as a potential therapy for cardioprotection after AMI.

Other advantages of the invention will become apparent to those of skill in the art upon reviewing the present specification.

### BRIEF DESCRIPTION OF THE DRAWINGS

Embodiments of the present invention will be described with reference to the accompanying drawings, wherein like reference numerals denote like parts, and in which:

FIG. 1 illustrates the protective effect of the NIMoEsh-Tat peptide on a primary cardiomyocyte culture from H₂O₂-induced cell damage. 10 μM NIMoEsh-Tat peptide (30 min pre-treatment+12 hrs post-treatment) was bath applied to a H₂O₂-treated (300 μM for 4 hrs) primary cardiocardiomyocyte culture and its protective efficacy was determined 12 hrs after H₂O₂ treatment using a variety of cell death assays: (a) MTT assay (Control: N=3; H₂O₂: N=6; H₂O₂+NIMoEsh-Tat:N=6; F(2,12)=10.05, P<0.01); (b) Cell apoptosis assay (Control: N=3; H₂O₂: N=4; H₂O₂+NIMoEsh-Tat:N=6; F(2,10)=56.67, P<0.001); (c) CK activity in the culture medium (Control: N=3; H₂O₂: N=6; H₂O₂+NIMoEsh-Tat:N=6; F(2,12)=20.70, P<0.001); (d) MDA activity in cultured cells (Control: N=3; H₂O₂: N=6; H₂O₂+NIMoEsh-Tat:N=6; F(2,12)=19.28, P<0.001); (e) LDH activity in the culture medium (Control: N=3; H₂O₂: N=6; H₂O₂+NIMoEsh-Tat: N=6; F(2,12)=16.10, P<0.001); (f) SOD activity in cultured cells (Control: N=3; H₂O₂: N=6; H₂O₂+NIMoEsh-Tat: N=6; F(2,12)=13.42, P<0.01). Data are presented as mean±S.E.M. The statistical difference between groups is determined by one-way ANOVA, followed by LSD post hoc test. *P<0.05, **P<0.01 and ***P<0.001 denote significant differences. n.s. denotes not significant.

FIG. 2 illustrates the protective effect of the NIMoEsh-Tat peptide against AMI-induced heart tissue infarct. (a) TTC staining of fresh rat hearts and (b) quantification bar graph showing that compared with the saline group, the NIMoEsh-Tat peptide reduced the percentage of heart tissue infarct after AMI (Saline group: N=10; NIMoEsh-Tat group: N=11; t(19)=3.71, P<0.01). Heart tissue infarct regions are highlighted by dotted lines in (a). For the heart function analysis in (c)-(h): the sham group: N=8; the saline group: N=7; the NIMoEsh-Tat peptide group: N=8. The sham group, the saline group and the NIMoEsh-Tat peptide group showed no significant differences in (c) the heart rate (F(2,20)=0.04, P=0.96) and (d) the systolic blood pressure (F(2,20)=1.74, P=0.20) after sham or AMI surgery. Compared with the saline group, the NIMoEsh-Tat peptide treatment group effectively rescued AMI-induced heart functional loss, as shown by a variety of assays: (e) left ventricular systolic pressure (F(2,20)=5.09, P<0.05); (f) left ventricular end diastolic pressure (F(2,20)=42.93, P<0.001); (g) +dp/dtmax (F(2,20)=27.98, P<0.001); (h) −dp/dtmax (F(2,20)=7.34, P<0.01). Data are presented as mean±S.E.M. The statistical difference between groups in (b) was determined by unpaired t test. The statistical difference between groups in (c)-(h) was determined by one-way ANOVA, followed by LSD post hoc test. **P<0.01 and ***P<0.001 denote significant differences. n.s. denotes not significant.

FIG. 3 illustrates the protective effect of the NIMoEsh-Tat peptide against AMI-induced heart damage and functional loss. Compared with saline control (N=5), the NIMoEsh-Tat peptide (N=5) reduced AMI-induced deficits in (c) stroke volume (after AMI: t(8)=−7.04, P<0.001) and (d) ejection fraction (after AMI: t(4)=−4.31, P<0.05), but not in (a) end-diastolic volume (after AMI: t(8)=−1.71, P=0.13) and (b) end-systolic volume (AMI: t(8)=1.30, P=0.23) in pigs. (e) quantification bar graph and (f) representative TTC staining images showing that compared with saline, the NIMoEsh-Tat peptide reduced AMI-induced heart infarct in pigs (t(8)=4.57, P<0.01). Heart tissue infarct regions are highlighted by dotted lines in (f). (g) NIMoEsh-Tat peptide-treated pig heart showed less fibrillation than that in the saline-treated pig heart. Scale bar in (g): 200 μm. Data are presented as mean±S.E.M. The statistical difference between groups was determined by unpaired t test. *P<0.05, **P<0.01 and ***P<0.001 denote significant differences. n.s. denotes not significant.

FIG. 4 illustrates unbiased treatment and animal selection. The heart rate (a), electrocardiogram (QRS duration (b), QRS voltage (c), T voltage (d) and ST voltage (e)), and body weight (f) were monitored before artery occlusion, after artery occlusion and after reperfusion. Pathological Q waves (b and c), increased T voltage (d) and increased ST voltage (e) suggested success of the AMI surgery in pigs. Both groups of pigs showed almost the same responses to the AMI surgery, indicating unbiased treatment and animal selection. The NIMoEsh-Tat group: N=5; the saline group: N=5. Data are presented as mean±S.E.M.

### DETAILED DESCRIPTION

As used herein, the term “NIMoEsh” refers to the following amino acid sequence:

[SEQ ID NO: 1]
WAAYRTHSVD

The present disclosure relates to a method of treating a disease or condition that is, or is associated with, acute myocardial infarction (AMI) in a subject in need thereof, the method comprising administering a therapeutically effective amount of a polypeptide comprising NIMoEsh to the subject. In one aspect of the present disclosure, the disease or condition is AMI.

The present disclosure also relates to a method of restoring heart function after AMI in a subject in need thereof, the method comprising administering a therapeutically effective amount of a polypeptide comprising NIMoEsh to the subject.

The present disclosure also relates to a method of reducing or preventing AMI-induced heart function loss in a subject in need thereof, the method comprising administering a therapeutically effective amount of a polypeptide comprising NIMoEsh to the subject.

The present disclosure also relates to a method of reducing AMI-induced heart tissue infarct in a subject in need thereof, the method comprising administering a therapeutically effective amount of a polypeptide comprising NIMoEsh to the subject.

The present disclosure also relates to a method of protecting cardiomyocytes against AMI-induced function loss in a subject in need thereof, the method comprising administering a therapeutically effective amount of a polypeptide comprising NIMoEsh to the subject.

The present disclosure also relates to use of a polypeptide comprising NIMoEsh to treat a disease or condition associated with AMI in a subject in need thereof.

The present disclosure also relates to use of a polypeptide comprising NIMoEsh to restore heart function after AMI in a subject in need thereof.

The present disclosure also relates to use of a polypeptide comprising NIMoEsh to reduce or prevent AMI-induced heart function loss in a subject in need thereof.

The present disclosure also relates to use of a polypeptide comprising NIMoEsh to reduce AMI-induced heart tissue infarct in a subject in need thereof.

The present disclosure also relates to use of a polypeptide comprising NIMoEsh to protect cardiomyocytes against AMI-induced function loss in a subject in need thereof.

Embodiments of these methods and uses may include any one of or a combination of any two or more of any of the following features:
the subject is a human;
the polypeptide is conjugated to a dat moiety;
the dat moiety is a protein transduction domain;
the protein transduction domain is HIV-1 Tat;
the polypeptide and dat moiety together have at least about 90%, or at least about 95%, or at least about 99% identity to the amino acid sequence of SEQ ID NO: 3, or the polypeptide and dat moiety together have the amino acid sequence of SEQ ID NO: 3;
the polypeptide is co-administered to the subject with one or more other active therapeutic ingredients, or the polypeptide is the only active therapeutic ingredient administered to the subject, or the subject has received another cardiovascular medication;
the polypeptide is administered in a pharmaceutical composition comprising one or more excipients;
the pharmaceutical composition is for systemic administration;
the pharmaceutical composition is for intravenous administration.

The present disclosure also relates to a polypeptide comprising NIMoEsh for any one of the above uses.

As used herein, ‘peptide’ or ‘polypeptide’ may be used interchangeably, and generally refer to a compound comprised of at least two amino acid residues covalently linked by peptide bonds or modified peptide bonds. However, when specifically used with reference to a specific SEQ ID NO, it is meant to comprise an amino acid sequence such as that represented by SEQ ID NO:1 or 3, wherein the peptide has cardioprotective activity. Modified peptide bonds may include for example peptide isosteres (modified peptide bonds) that may provide additional desired properties to the peptide, such as increased half-life. The amino acids comprising a peptide or polypeptide described herein may also be modified either by natural processes, such as posttranslational processing, or by chemical modification techniques which are well known in the art. Modifications can occur anywhere in a peptide, including the peptide backbone, the amino acid side-chains and the amino or carboxyl termini. It is understood that the same type of modification may be present in the same or varying degrees at several sites in a given peptide.

Amino acids are molecules containing an amine group, a carboxylic acid group and a side chain that varies between different amino acids. An amino acid may be in its natural form or it may be a synthetic amino acid. An amino acid may be described as, for example, polar, non-polar, acidic, basic, aromatic or neutral. A polar amino acid is an amino acid that may interact with water by hydrogen bonding at biological or near-neutral pH. The polarity of an amino acid is an indicator of the degree of hydrogen bonding at biological or near-neutral pH. Examples of polar amino acids include serine, proline, threonine, cysteine, asparagine, glutamine, lysine, histidine, arginine, aspartate, tyrosine and glutamate. Examples of non-polar amino acids include glycine, alanine, valine, leucine, isoleucine, methionine, phenylalanine, and tryptophan. Acidic amino acids have a net negative charge at a neutral pH. Examples of acidic amino acids include aspartate and glutamate. Basic amino acids have a net positive charge at a neutral pH. Examples of basic amino acids include arginine, lysine and histidine. Aromatic amino acids are generally nonpolar, and may participate in hydrophobic interactions. Examples of aromatic amino acids include phenylalanine, tyrosine and tryptophan. Tyrosine may also participate in hydrogen bonding through the hydroxyl group on the aromatic side chain. Neutral, aliphatic amino acids are generally nonpolar and hydrophobic. Examples of neutral amino acids include alanine, valine, leucine, isoleucine and methionine. An amino acid may be described by more than one descriptive category. Amino acids sharing a common descriptive category may be substitutable for each other in a peptide. An amino acid residue may be generally represented by a one-letter or three-letter designation, corresponding to the trivial name of the amino acid, in accordance with the following Table A. Amino acids comprising the peptides described herein will be understood to be in the L- or D-configuration. Amino acids described herein may be modified by methylation, amidation, acetylation or substitution with other chemical groups which may change the circulating half-life of the peptide without adversely affecting their biological activity.

It will be appreciated by a person of skill in the art that aspects of the individual amino acids in a polypeptide described herein may be substituted. Furthermore, it will be appreciated by a person of skill in the art that certain substitutions are more likely to result in retention of activity. For example, amino acids may be described as, for example, polar, non-polar, acidic, basic, aromatic or neutral. A polar amino acid is an amino acid that may interact with water by hydrogen bonding at biological or near-neutral pH. The polarity of an amino acid is an indicator of the degree of hydrogen bonding at biological or near-neutral pH. Examples of polar amino acids include serine, proline, threonine, cysteine, asparagine, glutamine, lysine, histidine, arginine, aspartate, tyrosine and glutamate. Examples of non-polar amino acids include glycine, alanine, valine leucine, isoleucine, methionine, phenylalanine, and tryptophan. Acidic amino acids have a net negative charge at a neutral pH. Examples of acidic amino acids include aspartate and glutamate. Basic amino acids have a net positive charge at a neutral pH. Examples of basic amino acids include arginine, lysine and histidine. Aromatic amino acids are generally nonpolar, and may participate in hydrophobic interactions. Examples of aromatic amino acids include phenylalanine, tyrosine and tryptophan. Tyrosine may also participate in hydrogen bonding through the hydroxyl group on the aromatic side chain. Neutral, aliphatic amino acids are generally nonpolar and hydrophobic. Examples of neutral amino acids include alanine, valine, leucine, isoleucine and methionine. An amino acid may be described by more than one descriptive category. Amino acids sharing a common descriptive category may be substitutable for each other in a peptide.

The term “identity” as used herein refers to the measure of the identity of sequence between two peptides. Identity can be determined by comparing a position in each sequence which may be aligned for purposes of comparison. For example, identity may be determined by the BLAST algorithm currently in use and which was originally described in Altschul et al. (1990) J. Mol. Biol. 215:403-410. The BLAST algorithm may be used with the published default settings. When a position in the compared sequence is occupied by the same amino acid, the molecules are considered to have shared identity at that position. The degree of identity between sequences is a function of the number of matching positions shared by the sequences and the degree of overlap between the sequences. Furthermore, when considering the degree of identity with SEQ ID NOs:1 or 3, it is intended that the equivalent number of amino acids be compared to SEQ ID NOs:1 or 3, respectively. Additional sequences (i.e. other than those corresponding to the 10 or 21 amino acids of SEQ ID NOs:1 or 3, respectively), are not intended to be considered when determining the degree of identity with SEQ ID NOs:1 or 3. The sequence identity of a given sequence may be calculated over the length of the reference sequence (i.e. SEQ ID NOs:1 or 3).

Nomenclature used to describe the peptides or polypeptides may follow the conventional practice where the amino group is presented to the left and the carboxy group to the right of each amino acid residue. In the sequences representing selected specific embodiments of the present invention, the amino- and carboxy-terminal groups, although not specifically shown, will be understood to be in the form they would assume at physiologic pH values, unless otherwise specified. In the amino acid structure formulae, each residue may be generally represented by a one-letter or three-letter designation, corresponding to the name of the amino acid, in accordance with the following Table A.

Table A Nomenclature and abbreviations of the 20 standard L-
amino acids commonly found in naturally occurring peptides

| | Three-letter | One-letter |
|---|---|---|
| Full name | abbreviation | abbreviation |
| Alanine | Ala | A |
| Cysteine | Cys | C |
| Aspartic acid | Asp | D |
| Glutamic acid | Glu | E |
| Phenylalanine | Phe | F |
| Glycine | Gly | G |
| Histidine | His | H |
| Isoleucine | Ile | I |
| Lysine | Lys | K |
| Leucine | Leu | L |
| Methionine | Met | M |
| Asparagine | Asp | N |
| Proline | Pro | P |
| Glutamine | Gln | Q |
| Arginine | Arg | R |
| Serine | Ser | S |
| Threonine | Thr | T |
| Valine | Val | V |
| Tryptophan | Trp | W |
| Tyrosine | Tyr | Y |

One or both, but usually one terminus of the peptide, may be substituted with a lipophilic group, usually aliphatic or aralkyl group, which may include heteroatoms. Chains may be saturated or unsaturated. Conveniently, commercially available aliphatic fatty acids, alcohols and amines may be used, such as caprylic acid, capric acid, lauric acid, myristic acid and myristyl alcohol, palmitic acid, palmitoleic acid, stearic acid and stearyl amine, oleic acid, linoleic acid, docosahexaenoic acid, etc. Preferred are unbranched, naturally occurring fatty acids between 14-22 carbon atoms in length. Other lipophilic molecules include glyceryl lipids and sterols, such as cholesterol. The lipophilic groups may be reacted with the appropriate functional group on the oligopeptide in accordance with conventional methods, frequently during the synthesis on a support, depending on the site of attachment of the oligopeptide to the support. Lipid attachment is useful where oligopeptides may be introduced into the lumen of the liposome, along with other therapeutic agents for administering the peptides and agents into a host.

Depending upon their intended use, particularly for administration to mammalian hosts, the subject peptides may also be modified by attachment to other compounds for the purposes of incorporation into carrier molecules, changing peptide bioavailability, extending or shortening half-life, controlling distribution to various tissues or the blood stream, diminishing or enhancing binding to blood components, and the like.

The exemplary peptides may further comprise a delivery and targeting (dat) moiety to assist in the transportation of the exemplary peptide across cell membranes. The term delivery and targeting (dat) moiety as used herein is meant to encompass any moiety that assists in delivering and/or targeting the peptides described herein to a target cell or tissue or within a target cell or within the cells of a target tissue. Furthermore, a dat moiety may “assist” in delivery and/or targeting by virtue of promoting the biological efficacy of the peptides described herein. Moieties that enable delivery or targeting of bioactive molecules into cells in a suitable manner so as to provide an effective amount, such as a pharmacologically effective amount, are known in the art. Optionally, the delivery and targeting (dat) moiety may be selected from one or more of: receptor ligands, protein transduction domains, micelles, liposomes, lipid particles, viral vectors, peptide carriers, protein fragments, or antibodies. Optionally, the protein transduction domain may be the cell-membrane transduction domain of HIV-1 Tat (Demarchi et al. (1996) J Virol. 70: 4427-4437). Other examples and related details of such protein transduction domains are described and known to those skilled in the art. The HIV-1 Tat cell-membrane transduction domain may form a fusion protein with an exemplary peptide described herein (for example, as in SEQ ID NO:3). These proteins may be produced through chemical synthesis, recombinant DNA, genetic and molecular engineering techniques known in the art.

In therapeutic applications, the compositions described herein may be administered to a subject suffering from one or more symptoms of a disease or condition, for example, a disease or condition associated with acute myocardial infarction (AMI). The composition described herein may be administered to a subject in an amount sufficient to cure or at least partially prevent or arrest the disease or condition and/or its complications or to help alleviate the symptoms associated therewith. An amount adequate to accomplish a treatment, cure or prophylactic treatment is defined as a “therapeutically effective dose” or “a therapeutically effective amount”. Amounts effective for this use will depend upon the severity of the disease or condition, the intended use (treatment, cure, prophylactic, alleviation of symptoms, etc.) and the general state of the subject's health. Single or multiple administrations of the compositions may be administered depending on the dosage and frequency as required and tolerated by the patient. A composition generally would provide a sufficient quantity of the active peptide or peptides described herein to effectively treat (for example, to at least ameliorate one or more symptoms) in the subject.

The concentration of peptide described herein can vary widely, and may be selected primarily based on fluid volumes, viscosities, body weight and the like in accordance with the particular mode of administration selected and the subject's needs. Concentrations, however, will typically be selected to provide dosages ranging from about 0.01 or 1 mg/kg/day to about 50 mg/kg/day and sometimes higher. It will be appreciated that such dosages may be varied to optimize a therapeutic regimen in a particular subject or group of subjects.

Additional active therapeutic ingredients may be administered to the subject along with or prior to the primary active agent, e.g., the exemplary peptides described herein. The exemplary peptide may be co-administered with another therapeutically active agent to enhance the therapeutic effect on the target cell or tissue by delivering a second compound with a similar or complementary activity. In one embodiment, such agents include, but are not limited to agents that reduce the risk of acute myocardial infarction (AMI) and/or complications thereof. Such agents include, but are not limited to, anti-coagulants (for example, Acenocoumarol, Coumatetralyl, Dicoumarol, Ethyl biscoumacetate, Phenprocoumon, Warfarin, Clorindione, Diphenadione, Phenindione, Tioclomarol, Bemiparin, Certoparin, Dalteparin, Enoxaparin, Nadroparin, Parnaparin, Reviparin, Tinzaparin, Fondaparinux, Idraparinux, Danaparoid, Sulodexide, Dermatan sulfate, Apixaban, Betrixaban, Edoxaban, Otamixaban, Rivaroxaban, Hirudin, Bivalirudin, Lepirudin, Desirudin, Argatroban, Dabigatran, Melagatran, Ximelagatran, REG1, Defibrotide, Ramatroban, Antithrombin III, and Drotrecogin alfa), anti-platelet drugs (for example, Abciximab, Eptifibatide, Tirofiban, Clopidogrel, Prasugrel, Ticlopidine, Ticagrelor, Beraprost, Prostacyclin, Iloprost, Treprostinil, Acetylsalicylic acid/Aspirin, Aloxiprin, Carbasalate calcium, Indobufen, Triflusal, Dipyridamole, Picotamide, Terutroban, Cilostazol, Dipyridamole, Triflusal, Cloricromen, Ditazole), and thrombolytic and fibrinolytic drugs (for example, tissue plasminogen activator (tPA) or recombinant tissue plasminogen activator (rtPA) such as Alteplase, Reteplase, Tenecteplase, Urokinase, Saruplase, Streptokinase, Anistreplase, Monteplase, Ancrod, Fibrinolysin, and Brinase), and the like or in combination with other cardioprotective agents.

Peptides may be prepared in a number of ways. Chemical synthesis of peptides is well known in the art. Solid phase synthesis is commonly used and various commercial synthetic apparatuses are available, for example automated synthesizers by Applied Biosystems Inc., Foster City, Calif.; Beckman; etc. Solution phase synthetic methods may also be used, particularly for large-scale productions.

Peptides may also be present in the form of a salt, generally in a salt form which is pharmaceutically acceptable. These include inorganic salts of sodium, potassium, lithium, ammonium, calcium, magnesium, iron, zinc, copper, manganese, and the like. Various organic salts of the peptide may also be made with, including, but not limited to, acetic acid, propionic acid, pyruvic acid, maleic acid, succinic acid, tartaric acid, citric acid, benozic acid, cinnamic acid, salicylic acid, etc. The prior examples serve as examples and are non-limiting.

### EXPERIMENTAL EXAMPLE

Embodiments of the present invention will be described with reference to the following exemplary information which should not be used to limit or construe the teachings described herein.

### Animals

Young Sprague Dawley rats (P1-2) and adult male Sprague Dawley rats (180-220 g) were purchased from B&K Universal Ltd in China. Adult rats were housed in plastic cages with free access to food and water and maintained in a temperature-controlled room (22-25° C.) with a 12/12 hr light/dark cycle. All experimental protocols were approved by the Second Military Medical University, and the methods were carried out in accordance with the approved guidelines and regulations. All efforts were made to minimize animal suffering and to reduce the number of animals used.

Chinese Bama miniature pigs (2-3 months; 7.81-10.43 Kg) were purchased from Wujiang Tianyu Biotech (Suzhou, China). Pigs were housed in stainless cages with free acess to food and water and maintained in a temperature-controlled room (18-26° C.) with a 12/12 hr light/dark cycle. All experimental protocols were approved by the contract research organization, JOINN Laboratories (Suzhou) (China, http://www.joinn-lab.com/), and the methods were carried out in accordance with approved guidelines and regulations. All efforts were made to minimize animal suffering and to reduce the number of animals used.

### Chemicals and Reagents

The following chemicals and reagents were used: Ketamine hydrochloride (Fujian Gutian Pharmaceutical, China, H35020148), diazepam (Henan Anyang Yikang Pharmaceutical, China, H41021491), saline (Shandong Hualu Pharmaceutical, China, H37022749), 2,3,5-triphenyl-tetrazolium chloride (TTC) (Sangon Biotech, China, CA25BA0012), urethane (Sinopharm Chemical Reagent, China, 20150908), DMEM (Hyclone, AB10155403), FBS (SAFC Biosciences, 8J0157), trypsin (Gibco, 1766146), type II collagenase (Sigma, 234155), 5′-Brdu (Bio Basic Inc, MC0325B2011Z), MTT (AMRESCO, LJ0628A5010J), Annexin V-FITC cytotoxicity kit (Beyotime Biotechnology, C1063), creatine kinase detection kit (Nanjing Jiancheng Bioengineering Institute, 20161130), lactate dehydrogenase detection kit (Nanjing Jiancheng Bioengineering Institute, 20161206), malondialdehyde detection kit (Nanjing Jiancheng Bioengineering Institute, 20161205), superoxide dismutase detection kit (Nanjing Jiancheng Bioengineering Institute, 20161205).

As used herein, the term “NIMoEsh” is referring to the following amino acid sequence: WAAYRTHSVD [SEQ ID NO: 1]. In the example described here, the NIMoEsh peptide (WAAYRTHSVD) is conjugated to a TAT protein transduction domain (YGRKKRRQRRR; SEQ ID NO: 2). The NIMoEsh-Tat peptide (WAAYRTHSVD-YGRKKRRQRRR; SEQ ID NO: 3) was chemically synthesized in the peptide facility at the Center for Brain Health of University of British Columbia using the Prelude peptide synthesizer (Protein Technologies Inc.).

### Buffers and Media

Cell digestion buffer (EDTA free) contained 0.05% trypsin and 0.5 mg/ml type II collagenase. Cell culture medium contained 15% FBS, 1% Penicillin-Streptomycin, 1% 0.1 mM Brdu and 83% DMEM.

### Peptide Preparation and Treatment

The peptide solution was prepared fresh before each use by dissolving dry peptide powder into sterile water or saline. For cell culture experiments, the peptide stock solution was dissolved in the culture medium to the desired concentration. For animal experiments, the peptide solution was intravenously injected.

### Primary Cardiomyocyte Culture

Hearts were removed from P1-2 Sprague Dawley rats and cut into 1 mm³ cubes. The heart tissue was then digested in the cell digestion buffer at 37° C. for 4 min and centrifuged at 200 rpm for 1 min to remove the supernatant. The pellet was digested in the cell digestion buffer again at 37° C. until it was fully digested and then centrifuged at 200 rpm for 1 min to remove the debris. The sample was mixed with FBS to neutralize trypsin and collagenase and then centrifuged at 1000 rpm for 5 min to remove the supernatant. Cardiomyocytes were isolated from the sample by differential adhesion and then cultured in plates in the cell culture medium. Primary cardiomyocyte cultures were maintained in the 37° C. incubator with 95% O₂ and 5% CO₂ for 5 days before being used for experiments.

### Cell Death Assays

Cell death of primary cardiomyocyte culture was measured using a variety of assays. MTT and the activity of creatine kinase, lactate dehydrogenase, malondialdehyde and superoxide dismutase were measured using commercial kits and according to manufacturer's instructions. Cell apoptosis assays were conducted by treating primary cardiomyocyte culture with the Annexin V-FITC cytotoxicity kit, followed by flow cytometry (BD, FACSlalibur) to determine the apoptosis percentage.

### Rat Model of AMI

Rats were subjected to AMI by ligation of the left anterior descending coronary artery as described previously (Yu, J. G. et al. Acta Pharmacol Sin 34, 1508-1514, doi:10.1038/aps.2013.147 (2013)). Briefly, rats were anesthetized with 100 mg/kg ketamine hydrochloride and 10 mg/kg diazepam by intraperitoneal injection and then received artificial ventilation using a respirator (Shanghai Alcott Biotech, China, ALC-V8) with a tidal volume of 20 mL and a respiratory rate of 60/min. The heart was exteriorized through the 4th intercostal space and the left anterior descending coronary artery was ligated using a 6-0 suture. Rats were placed back in their home cages after the incision was closed. Sham rats underwent the same protocol except without ligation of the coronary artery.

### Pig Model of Transient AMI

Pigs were subject to transient AMI by temporarily occluding the left circumflex coronary artery as described previously (Ichimura, K. et al. PLoS One 11, e0162425, doi:10.1371/journal.pone.0162425 (2016)). Briefly, pigs were anesthetized with ketamine hydrochloride (10 mg/kg, intramuscular) and were maintained under anesthesia with isoflurane using a ventilator (SN23402, Hallowell engineering and manufacturing corporation, US) (tidal volume: 80 mL; respiratory rate: 20/min). A left thoracotomy was performed and a pneumatic cuff occluder was placed at the proximal end of the left circumflex coronary artery. Pigs were placed back to their home cages after the incision was closed. On the experiment day, the left circumflex coronary artery was occluded for 60 min to induce AMI by inflating the cuff. Then the cuff was deflated and blood reperfusion started. Jacketed external telemetry (Data Science International Inc., US) was used to monitor ECG signals in pigs before artery occlusion and until 10 hours after blood reperfusion.

### TTC Staining and Measurement of Heart Infarct

Rats were sacrificed and their hearts were removed and weighed on an electronic scale. After snap freezing in a −20° C. freezer for 20 min, hearts were sliced into 5 equally sized pieces and placed in the 0.5% TTC solution at 37° C. for 5 min. Infarct tissue, as identified as tissue that was white in color, was separated from the heathy tissue, as identified as tissue that was red in color. Infarct tissue was then weighed on an electronic scale and the percentage of heart infarct was calculated by dividing the weight of infarct tissue by the weight of the whole heart.

Pigs were sacrificed and the hearts were removed. After rinsing with saline, hearts were perfused with 37° C. 1% TTC and then sliced into 6 pieces (5 mm thickness). Heart slices were placed in the 1% TTC solution at 37° C. for 5-10 min and then fixed with 10% formalin solution. Heart slice images were taken and then analyzed using NIH Image J software. Heart infarct volume in each slice=(infarct area on one side of the slice+infarct area on the other side of the slice)/2×thickness (5 mm). The heart infarct percentage was calculated by dividing the volume of infarct tissue by the volume of the left ventricle.

### Heart Function Measurement

Hemodynamic assessments in rats were conducted as described by Yu et al. (Acta Pharmacol Sin 34, 1508-1514, doi:10.1038/aps.2013.147 (2013)). Briefly, rats were anesthetized with 1.25 g/kg 25% urethane and a polyethylene catheter connected to a pressure transducer (Powerlab 30, ADlnstruments) was inserted into the right carotid artery and then advanced into the left ventricle cavity. The mean heart rate, systolic blood pressure, left ventricular systolic pressure (LVSP), left ventricular end diastolic pressure (LVEDP), and maximal rates of pressure rise (+dp/dtmax) and decline (−dp/dtmax) were recorded for 30 min and then analyzed.

Transthoracic echocardiography in pigs were conducted using Portable Color Doppler (S8 Exp, Shenzhen SonoScape Medical Corp, China) after pigs were anesthetized with telazol (10 mg/kg, intramuscular) and isoflurane. The end-diastolic volume (EDV), end-systolic volume (ESV), stroke volume (SV), and ejection fraction (EF) were recorded.

### Histological Analysis of Pig Hearts

The 2^{nd} slice from each pig heart was processed for H & E staining and then histological analysis. Levels of heart pathology (atrial fibrillation, necrosis, bleeding, inflammation, granuloma and pericarditis) were classified into 4 categories for comparison: slight (+; almost invisible), mild (++; visible but very small), moderate (+++; visible and quantifiable) and severe (++++; extensive damage).

### Statistical Analysis

Cell cultures and animals were randomly assigned into experimental treatment groups and data were analyzed using SPSS software and expressed as mean±S.E.M. Data were analyzed by one-way ANOVA, followed by LSD post hoc test, or by unpaired t test. *P<0.05, **P<0.01, and ***P<0.001 were considered as significant differences.

### Results

### Cardioprotective Efficacy of the NIMoEsh-Tat Peptide in an in Vitro Model of AMI

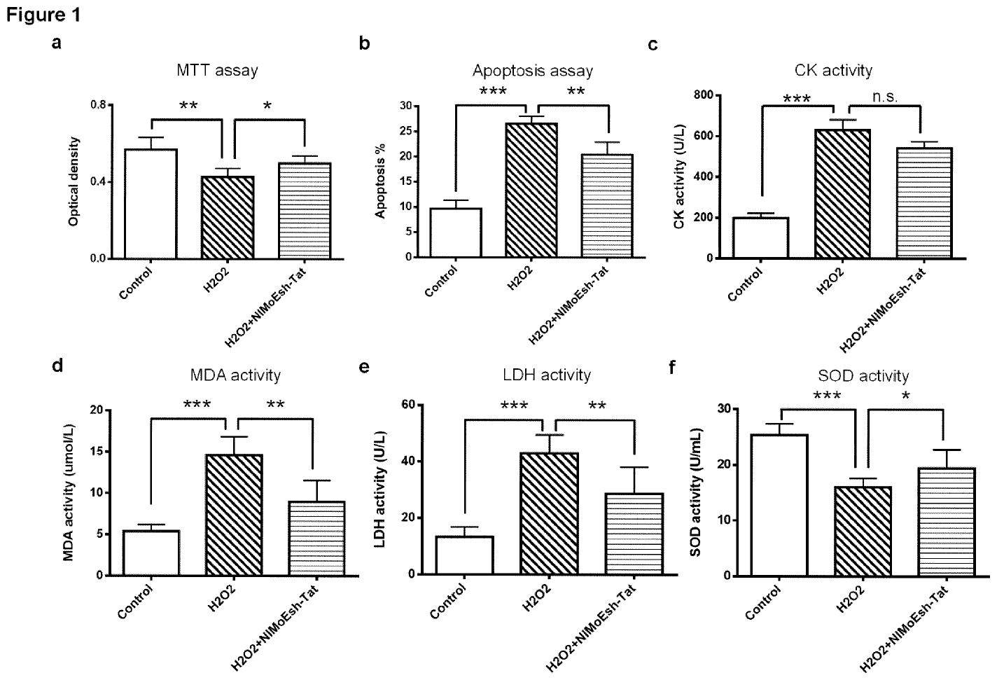

Primary cardiomyocyte cultures were treated with 300 uM H₂O₂ for 4 hours to induce oxidative stress. Then, cardiomyocytes were placed back in the normal culture medium for 12 hours before cell death analysis. 30 min before H₂O₂ treatment and during the 12 hour-recovery period, some cardiomyocytes were treated with 10 uM NIMoEsh-Tat peptide. Compared with the untreated control, H₂O₂ treatment induced cell death, as shown by the MTT assay (FIG. 1a) and the cell apoptosis assay (FIG. 1b), increased the activities of cardiomyocyte damage markers, as shown by assays of creatine kinase (CK, FIG. 1c), malondialdehyde (MDA, FIG. 1d) and lactate dehydrogenase (LDH, FIG. 1e), and decreased the activity of superoxide dismutase (SOD), a pro-survival protein marker (FIG. 1f). Compared with the H₂O₂ group, the NIMoEsh-Tat peptide effectively reduced H₂O₂-induced cell death, as shown in FIG. 1a-b and FIG. 1d-f. Although the NIMoEsh-Tat peptide did not fully rescue H₂O₂-induced increase of CK activity, there was a trend of decreased CK activity in the peptide treated group compared with the H₂O₂ group (FIG. 1c). These results suggest cardioprotective efficacy of the NIMoEsh-Tat peptide against oxidative stress-induced cardiomyocyte death.

### The NIMOEsh-Tat Peptide Reduces Heart Damage and Restores Heart Function After AMI in Rats

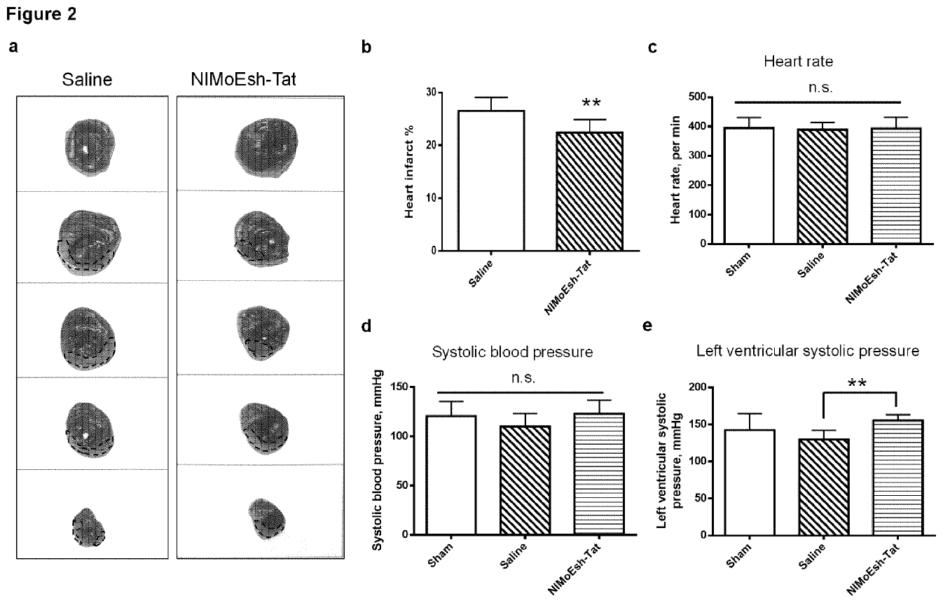

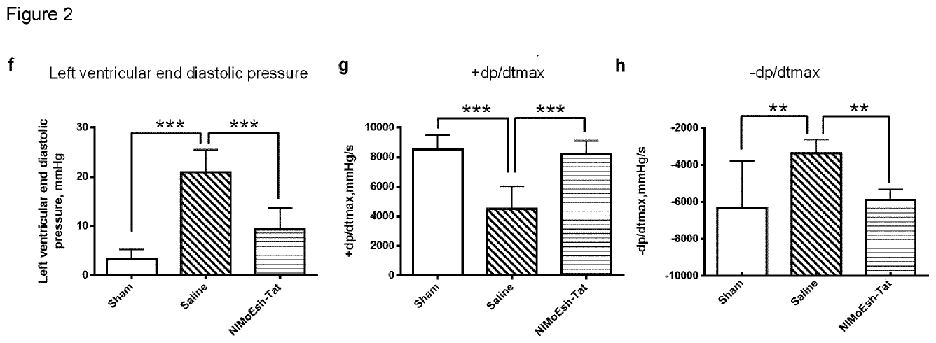

The cardioprotective efficacy of the NIMoEsh-Tat peptide in a well-established rat model of AMI (Yu, J. G. et al. Acta Pharmacol Sin 34, 1508-1514, doi:10.1038/aps.2013.147 (2013)) was investigated. 20 mg/kg NIMoEsh-Tat or saline control was intravenously (i.v.) injected into rats immediately after ligation of the left anterior descending coronary artery. Four hours later, hearts were removed for TTC staining and infarct percentage measurement. As shown in FIG. 2a-b, AMI surgery induced severe tissue infarct in the hearts of saline treated rats, and this surgery-induced heart damage was significantly reduced by NIMoEsh-Tat treatment, suggesting cardioprotective efficacy of the NIMoEsh-Tat peptide.

The long-term protective effects of the NIMoEsh-Tat peptide against AMI were also investigated. Rats that had ligation of the left anterior descending coronary artery received 3 i.v. injections of the NIMoEsh-Tat peptide (20 mg/kg) or saline at 0 hours, 24 hours and 48 hours after surgery. Rats receiving sham surgery were used as control. Heart function was measured 4 weeks after the last peptide injection using several well-characterized parameters (Yu, J. G. et al. Acta Pharmacol Sin 34, 1508-1514, doi:10.1038/aps.2013.147 (2013).) Compared with the sham group, neither saline or NIMoEsh-Tat peptide changed the heart rate (FIG. 2c) and the systolic blood pressure (FIG. 2d) in rats after AMI. Compared with the sham group, saline treated rats showed decreases in left ventricular systolic pressure (FIG. 2e) and in the maximal rates of pressure rise (+dp/dtmax) (FIG. 2g) and decline (−dp/dtmax) (FIG. 2h), and increases in left ventricular end diastolic pressure (FIG. 2f) after AMI, suggesting compromised heart function after AMI. The NIMoEsh-Tat peptide treatment rescued heart function after AMI compared with the saline group (FIG. 2e-h).

### The NiMoEsh-Tat Peptide Reduces Heart Infarct and Restores Heart Function After Transient AMI in Mini Pigs

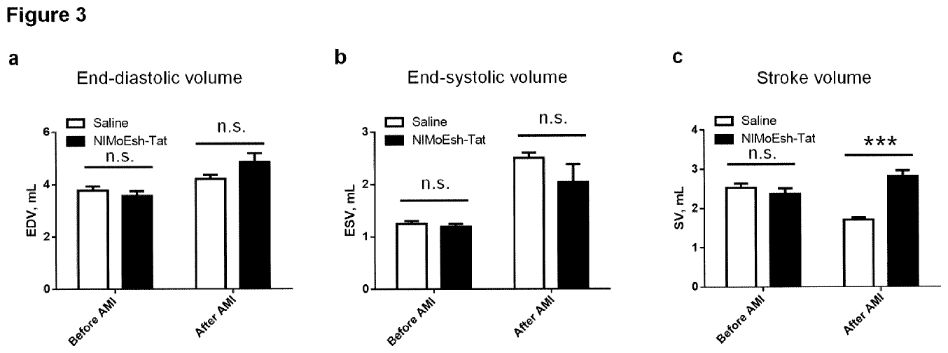

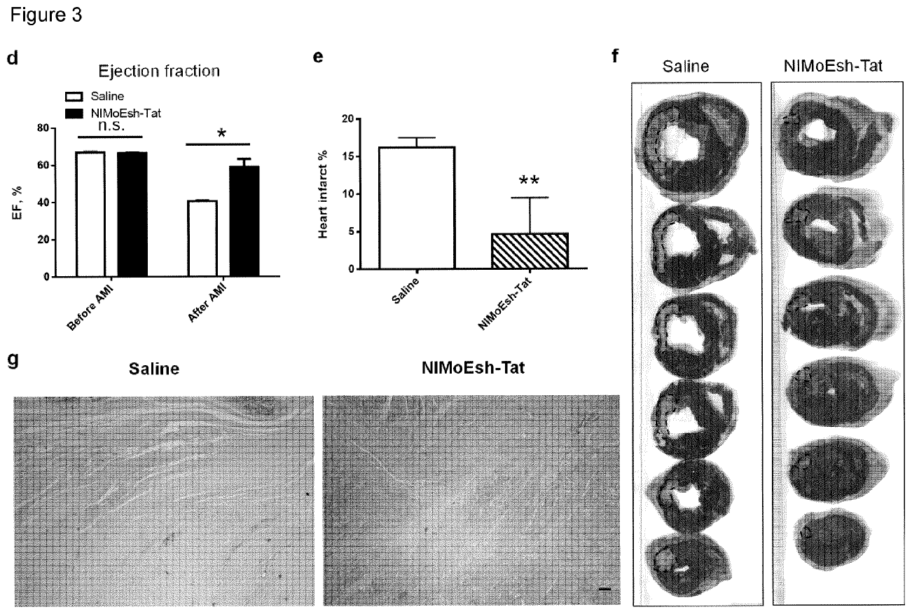

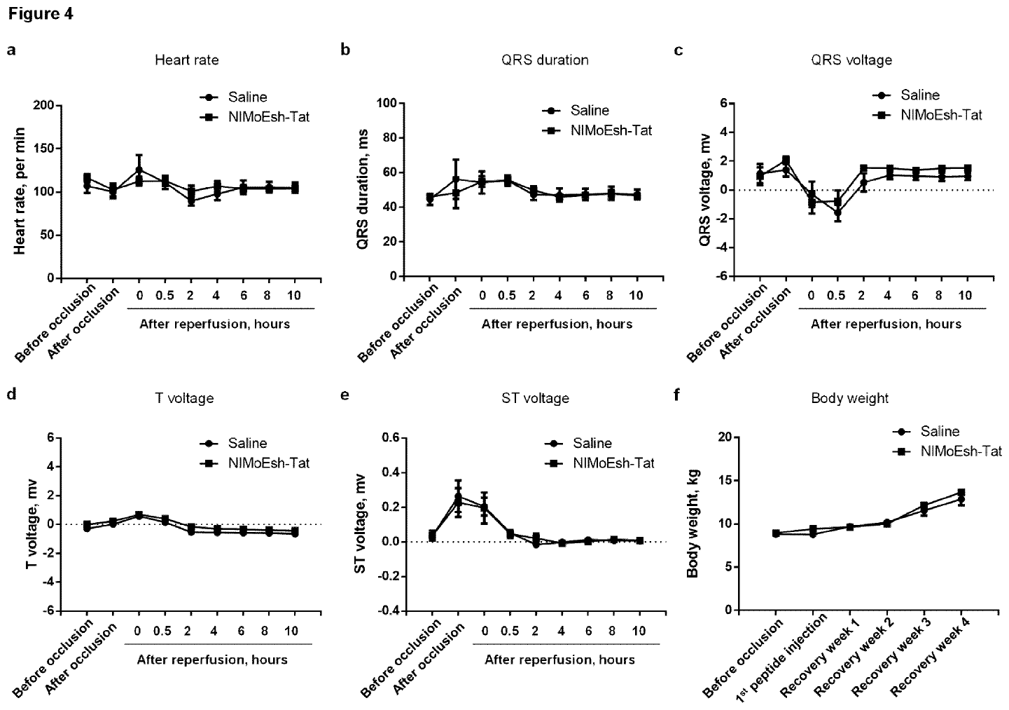

The cardioprotective efficacy of the NIMoEsh-Tat peptide was also investigated in Chinese Bama miniature pigs. Transient AMI was induced in these pigs by occluding the left circumflex coronary artery for 1 hour, followed by blood reperfusion. Pigs received one i.v. injection of the NIMoEsh-Tat peptide (2 mg/kg) or saline 40 min after occlusion and another i.v. injection 24 hours after reperfusion. The pigs were evaluated for heart function on day 30 before they were sacrificed for histological analysis. Both groups of pigs had the same level of artery occlusion during AMI surgery and also showed the same level of body weight recovery after AMI (FIG. 4). As shown in FIG. 3, the NIMoEsh-Tat peptide rescued AMI-induced function deficits in stroke volume (SV, FIG. 3c) and ejection fraction (EF, FIG. 3d). TTC staining of pig heart slices also revealed that the NIMoEsh-Tat peptide reduced the volume of AMI-induced heart infarct compared with the saline control (FIGS. 3e and 3f). This is consistent with the histological studies of hearts (FIG. 3g and Table 1), which suggests that the NIMoEsh-Tat peptide reduced AMI-induced heart pathology, such as atrial fibrillation and pericarditis, compared with control.

TABLE 1

Gender
Male
Group

| | Saline | NIMoEsh-Tat |
|---|---|---|
| Dose (mg/kg) | 0 | 2 |
| N number | 5 | 5 |

Atrial fibrillation

| | | |
|---|---|---|
| Total | 5 | 5 |
| + | 0 | 2 |
| ++ | 0 | 2 |
| +++ | 5 | 1 |

Necrosis

| | | |
|---|---|---|
| Total | 5 | 4 |
| | 0 | 4 |
| ++ | 5 | 0 |

Bleeding

| | | |
|---|---|---|
| Total | 5 | 4 |
| + | 5 | 4 |

Inflammation

| | | |
|---|---|---|
| Total | 5 | 4 |
| + | 4 | 4 |
| ++ | 1 | 0 |

Granuloma

| | | |
|---|---|---|
| Total | 2 | 1 |
| + | 2 | 1 |

Pericarditis

| | | |
|---|---|---|
| Total | 2 | 0 |
| + | 1 | 0 |
| | 1 | 0 |

Table 1 provides a summary of pathology observed under microscope. The NIMoEsh-Tat peptide-treated pigs showed much less AMI-induced pathology compared with the saline controls.

### Discussion

NIMoEsh-Tat not only protected cardiomyocytes against cell death in primary cultures of cardiomyocytes, but also protected against AMI-induced heart infarct and functional loss in both rats and pigs. While not wishing to be bound by any particular theory or mode of action, the results described herein suggest that the interaction between zD17 and JNK may play a role, at least in part, in inducing cell death in the heart and therefore the cardioprotective effects of NIMoEsh-Tat described herein may be attributed, at least in part, to the blocking of the interaction between zD17 and JNK during cell stress.

A potential concern about peptide therapeutics may be their short half-lives. Once in the body, peptides without chemical modifications can be prone to enzymatic digestion. This can be problematic for treatment of chronic diseases, such as Alzheimer's disease, Parkinson's disease and hypertension, because patients may require multiple injections per day. However, for acute indications, a small number of injections may be sufficient for disease treatment, making the short half-life of peptides less of a concern. This is supported by the results described herein. As shown in FIG. 2, one i.v. injection of the NIMoEsh-Tat peptide was sufficient to protect rats against AMI-induced heart tissue damage and one injection per day for 3 days was sufficient to effectively protect the heart against AMI-induced function loss. In FIG. 3 and Table 1, two i.v. injections of the NIMoEsh-Tat peptide were sufficient to induce cardioprotection in pigs after AMI.

While this invention has been described with reference to illustrative embodiments and examples, the description is not intended to be construed in a limiting sense. Thus, various modifications of the illustrative embodiments, as well as other embodiments of the invention, will be apparent to persons skilled in the art upon reference to this description. It is therefore contemplated that the appended claims will cover any such modifications or embodiments.

All publications, patents and patent applications referred to herein are incorporated by reference in their entirety to the same extent as if each individual publication, patent, or patent application was specifically and individually indicated to be incorporated by reference in its entirety.

SEQUENCE LISTING

SEQ ID NO: 1 (NlMoEsh): WAAYRTHSVD
SEQ ID NO: 2 (HIV-1 Tat protein transduction domain): YGRKKRRQRRR
SEQ ID NO: 3 (NlMoEsh-Tat peptide): WAAYRTHSVDYGRKKRRQRRR

1. A method of treating a disease or condition that is, or is associated with, acute myocardial infarction (AMI) in a subject in need thereof, the method comprising administering a therapeutically effective amount of a polypeptide comprising NIMoEsh to the subject.

2. The method of claim 1, wherein the disease or condition is AMI.

3. A method of restoring heart function after acute myocardial infarction (AMI) in a subject in need thereof, the method comprising administering a therapeutically effective amount of a polypeptide comprising NIMoEsh to the subject.

4. A method of reducing or preventing acute myocardial infarction (AMI)-induced heart function loss in a subject in need thereof, the method comprising administering a therapeutically effective amount of a polypeptide comprising NIMoEsh to the subject.

5. A method of reducing acute myocardial infarction (AMI)-induced heart tissue infarct in a subject in need thereof, the method comprising administering a therapeutically effective amount of a polypeptide comprising NIMoEsh to the subject.

6. A method of protecting cardiomyocytes against acute myocardial infarction (AMI)-induced function loss in a subject in need thereof, the method comprising administering a therapeutically effective amount of a polypeptide comprising NIMoEsh to the subject.

7. The method of claim 1, wherein the subject is a human.

8. The method of claim 1, wherein the polypeptide is conjugated to a delivery and targeting (dat) moiety.

9. The method of claim 8, wherein the dat moiety is the HIV-1 Tat protein transduction domain.

10. The method of claim 8, wherein the polypeptide and dat moiety together have at least about 90%, or at least about 95%, or at least about 99% identity to the amino acid sequence of SEQ ID NO: 3.

11. The method of claim 10, wherein the polypeptide and dat moiety together have the amino acid sequence of SEQ ID NO: 3.

12. The method of claim 1, wherein the polypeptide is co-administered to the subject with one or more other active therapeutic ingredients.

13. The method of claim 1, wherein the polypeptide is the only active therapeutic ingredient administered to the subject.

14. The method of claim 1, wherein the subject has received another cardiovascular medication.

15. The method of claim 1, wherein the polypeptide is administered in a pharmaceutical composition comprising one or more excipients.

16. The method of claim 15, wherein the pharmaceutical composition is for systemic administration.

17. The method of claim 16, wherein the pharmaceutical composition is for intravenous administration.

18-51. (canceled)
